(19) United States
(12) Patent Application Publication
(10) Pub. No.: US 2021/0407680 A1
(43) Pub. Date: Dec. 30, 2021

(54) SYSTEMS AND METHODS FOR MACHINE LEARNING MODELS FOR EXPERTISE MAPPING

(71) Applicant: Grand Rounds, Inc., San Francisco, CA (US)

(72) Inventors: Jyotiwardhan PATIL, San Francisco, CA (US); Matthew PANCIA, San Francisco, CA (US); Robert SHARP, San Francisco, CA (US); Ricardo PINHO, San Francisco, CA (US); Helena WANG, San Francisco, CA (US); Nathaniel FREESE, San Francisco, CA (US)

(73) Assignee: Grand Rounds, Inc., San Francisco, CA (US)

(21) Appl. No.: 17/364,653

(22) Filed: Jun. 30, 2021

Related U.S. Application Data
(60) Provisional application No. 63/046,683, filed on Jun. 30, 2020.

(51) Int. Cl. G16H 50/20; G16H 40/20; G16H 10/60

(52) U.S. Cl. CPC G16H 50/20; G16H 10/60; G16H 40/20

(57) ABSTRACT

Methods, systems, and computer-readable media for determining the expertise of service providers to match with users utilizing a service provider search system. The method identifies searched conditions and determine associated codes. The method next determines procedures provided by service providers associated with codes. The method then normalizes codes associated with conditions and selects a subset of them based on the popularity of procedures associated with the codes. The method finally utilizes a machine learning model to translate the subset of codes to topics and calculates similarity metric between the topics and the service providers and tunes the threshold of the metric. The method then an using the tuned threshold outputs a service provider based on a query to a service provider search system.

### CROSS-REFERENCE TO RELATED APPLICATIONS

This application claims priority to U.S. Provisional Application No. 63/046,683, filed on Jun. 30, 2020, the entirety of which is hereby incorporated by reference.

### BACKGROUND

An ever increasing amount of data and data sources are now available to researchers, analysts, organizational entities, and others. This influx of information allows for sophisticated analysis but, at the same time, presents many new challenges for sifting through the available data and data sources to locate the most relevant and useful information. As the use of technology continues to increase, so, too, will the availability of new data sources and information.

Because of the abundant availability of data from a vast number of data sources, determining the optimal values and sources for use presents a complicated problem difficult to overcome. Accurately utilizing the available data can require both a team of individuals possessing extensive domain expertise as well as many months of work to evaluate the outcomes. The process can involve exhaustively searching existing literature, publications, and other available data to identify and study relevant data sources that are available both privately and publicly.

While this approach can often provide effective academic analysis, applying these types of analytical techniques to domains requiring accurate results obtainable only through time and resource intensive research is incompatible with modern applications' demands. For example, the developed process for evaluating outcomes may not line up with specific circumstances or individual considerations. In this scenario, applying the process requires extrapolation to fit the specific circumstances, dilute the process's effectiveness, or require spending valuable time and resources to modify the process. As a result, processes developed in this way typically provide only generalized guidance insufficient for repurposing in other settings or by other users. As more detailed and individualized data becomes available, demand for the ability to accurately discern relevant data points from the sea of available information, and efficiently apply that data across thousands of personalized scenarios increases.

### SUMMARY

Certain embodiments of the present disclosure relate to a non-transitory computer readable medium, including instructions that when executed by one or more processors cause a system to perform a method. The method may include identifying conditions searched in a service provider search system; determining codes associated with the identified conditions, wherein each condition of the identified conditions is associated with one or more codes; determining procedures provided by service providers available through the service provider search system, wherein each service provider of the services providers available through the service provider search system provides one or more procedures, wherein the procedures are associated with the determined codes; normalizing the one or more codes associated with condition of the identified conditions; selecting a subset of codes of the determined codes, wherein the selection is based on the popularity of procedures associated with the codes; utilizing a machine learning model to translate the selected subset of codes to topics; determining a similarity metric between the topics and the service providers, wherein service providers are those whose procedures are associated with code; tuning threshold on the similarity metric; and providing, using the tuned threshold, an output of a service provider based on a query by a user utilizing the service provider search system.

According to some disclosed embodiments, identifying conditions may further include processing the historical information of use of the plurality of service providers.

According to some disclosed embodiments, selecting the subset of codes may further include selecting the code for the service providers with more probability to treat than average probability of a set of similar service providers.

According to some disclosed embodiments, selecting the subset of codes may further include identifying a subset of procedures that have most impact on outcome; and selecting the codes associated with the identified subset of treatment.

According to some disclosed embodiments, determining procedures provided by service providers may further include determining volume of each treatment of the procedures provided by each service provider of the one or more service providers.

According to some disclosed embodiments, the machine learning model is a topical model.

According to some disclosed embodiments, determining a similarity metric between the topics and the service providers available through the service provider search system may further include determining expertise requirement of the user of the service provider search system, wherein the experiment requirement is based on service provider usage history of the user; and determining a service provider with expertise level matching the expertise requirement.

According to some disclosed embodiments, the method may further include determining the specialty of the service providers; selecting the service provider with specialties matching the query, wherein the procedures associated with a specialty match the procedures associated with a condition presented in the query.

According to some disclosed embodiments, determining the specialty of service providers further include executing a machine learning model for each specialty, wherein a machine learning model is input the encounters of the service providers with the users of the service provider search system.

According to some disclosed embodiments, the method may further include assigning default specialty labels for the service providers provided by the third-party database.

According to some disclosed embodiments, tuning the threshold on the similarity metric may further include improving recall rate of similar set of service providers for similar set of user queries.

According to some disclosed embodiments, tuning the threshold on the similarity metric may further include improving precision rate of same set of service providers for similar set of user queries.

According to some disclosed embodiments, improving the precision rate of the same set of service providers includes maintaining the same order of the service providers.

According to some disclosed embodiments, the method may further include receiving queries for specific services.

According to some disclosed embodiments, the method may further include processing historic data from past; determining procedures performed by a service provider to handle a condition; generating a binary label for each condition based on the procedures; and building a machine learning model; and outputting probability of a service provider can handle a condition.

Certain embodiments of the present disclosure relate to a method performed by a system for determining the expertise of service providers to match with users utilizing a service provider search system. The method may include identifying conditions searched in a service provider search system; determining codes associated with the identified conditions, wherein each condition of the identified conditions is associated with one or more codes; determining procedures provided by service providers available through the service provider search system, wherein each service provider of the services providers available through the service provider search system provides one or more procedures, wherein the procedures are associated with the determined codes; normalizing the one or more codes associated with condition of the identified conditions; selecting a subset of codes of the determined codes, wherein the selection is based on the popularity of procedures associated with the codes; utilizing a machine learning model to translate the selected subset of codes to topics; determining a similarity metric between the topics and the service providers, wherein service providers are those whose procedures are associated with code; tuning threshold on the similarity metric; and providing, using the tuned threshold, an output of a service provider based on a query by a user utilizing the service provider search system.

Certain embodiments of the present disclosure relate to a system for determining the expertise of service providers to match with users utilizing a service provider search system. The system includes one or more processors executing processor-executable instructions stored in one or more memory devices to perform a method. The method may include identifying conditions searched in a service provider search system; determining codes associated with the identified conditions, wherein each condition of the identified conditions is associated with one or more codes; determining procedures provided by service providers available through the service provider search system, wherein each service provider of the services providers available through the service provider search system provides one or more procedures, wherein the procedures are associated with the determined codes; normalizing the one or more codes associated with condition of the identified conditions; selecting a subset of codes of the determined codes, wherein the selection is based on the popularity of procedures associated with the codes; utilizing a machine learning model to translate the selected subset of codes to topics; determining a similarity metric between the topics and the service providers, wherein service providers are those whose procedures are associated with code; tuning threshold on the similarity metric; and providing, using the tuned threshold, an output of a service provider based on a query by a user utilizing the service provider search system.

### BRIEF DESCRIPTION OF THE DRAWINGS

The accompanying drawings, which are incorporated in and constitute a part of this specification, illustrate several embodiments and, together with the description, serve to explain the disclosed principles. In the drawings:

FIG. 1 is a block diagram showing various exemplary components of a specialization system for determining expertise of service providers, according to some embodiments of the present disclosure.

FIG. 2 is a block diagram of an exemplary search engine 200, according to some embodiments of the present disclosure.

FIG. 3 illustrates a schematic diagram of an exemplary server of a distributed system, according to some embodiments of the present disclosure.

FIG. 4 is a flowchart showing an exemplary method for determining exact expertise of a service provider, according to some embodiments of the present disclosure.

FIG. 5 is a flowchart showing an exemplary method for generating expertise of a service provider, according to some embodiments of the present disclosure.

FIG. 6 is a flowchart showing an exemplary method for generating specialties of a service provider, according to some embodiments of the present disclosure.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are neither constrained to a particular order or sequence nor constrained to a particular system configuration. Additionally, some of the described embodiments or elements thereof can occur or be performed simultaneously, at the same point in time, or concurrently. Reference will now be made in detail to the disclosed embodiments, examples of which are illustrated in the accompanying drawings. Unless explicitly stated, sending and receiving as used herein are understood to have broad meanings, including sending or receiving in response to a specific request or without such a specific request. These terms thus cover both active forms, and passive forms, of sending and receiving.

The embodiments described herein provide technologies and techniques for evaluating large numbers of data sources and vast amounts of data used in the creation of a machine learning model. These technologies can use information relevant to the specific domain and application of a machine learning model to prioritize potential data sources. Further, the technologies and techniques herein can interpret the available data sources and data to extract probabilities and outcomes associated with the machine learning model's specific domain and application. The described technologies can synthesize the data into a coherent machine learning model, that can be used to analyze and compare various paths or courses of action.

These technologies can efficiently evaluate data sources and data, prioritize their importance based on domain and circumstance specific needs, and provide effective and accurate predictions that can be used to evaluate potential courses of action. The technologies and methods allow for the application of data models to personalized circumstances. These methods and technologies allow for detailed evaluation that can improve decision making on a case-by-case basis. Further, these technologies can evaluate a system where the process for evaluating outcomes of data may be set up easily and repurposed by other uses of the technologies.

Technologies may utilize machine learning models to automate the process and predict responses without human intervention. The performance of such machine learning models is usually improved by providing more training data. A machine learning model's prediction quality is evaluated manually to determine if the machine learning models need further training. Embodiments of these technologies described can help improve machine learning model predictions using the quality metrics of predictions requested by a user.

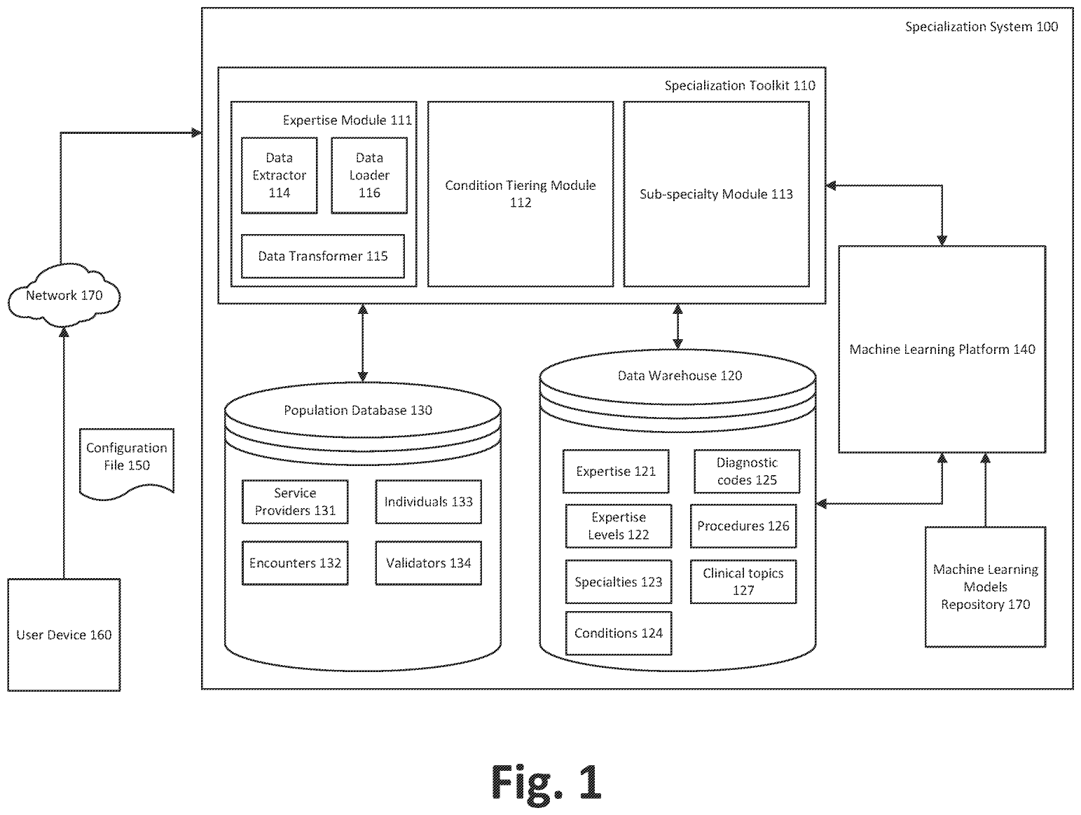

FIG. 1 is a block diagram showing various exemplary components of a specialization system 100 for determining expertise of service providers, according to some embodiments of the present disclosure. Expertise determination may include confirmation of a service provider expertise in performing work to handle certain conditions or performing certain procedures as part of handling certain conditions. A service provider may be regarded to handle a condition if they have past experience working on a condition. In some embodiments, a service provider may need to succeed in performing work on a condition to be regarded as having the ability to handle a condition. In some embodiments, a service provider is regarded to have the ability to handle a condition if they have not referred another service provider.

A service provider's expertise may include level of expertise of service providers as defined by a user of specialization system 100. A user of specialization system 100 may define levels of expertise of service providers using a text configuration file. Service provider expertise levels may be defined based on the effectiveness of the work performed by a service provider to handle conditions of concern. In some embodiments, service provider's expertise may include various specialties gained by the service provider through formal education and training. Specialization system 100 may determine various expertise in the form of expertise confirmation, levels of expertise, and specialty of training and education to help identify the relevant service providers for handling an identified condition in the most effective manner. Specialization system 100 may also consider other factors when identifying relevant service providers, such as cost, travel distance, and other individual preferences, etc.

As illustrated in FIG. 1, specialization system 100 may include specialization toolkit 110 to evaluate various expertise of service providers and data warehouse 120 to store the various determined expertise of service providers. Specialization toolkit 110 may help determine expertise of service providers using data from population database 130. Population database 130 may aid in determining expertise based on service providers (e.g., service providers 131) encounters (e.g., encounters 132) with individuals (e.g., individuals 133). A detailed description of data warehouse 120 and population database 130 and their contents is provided below.

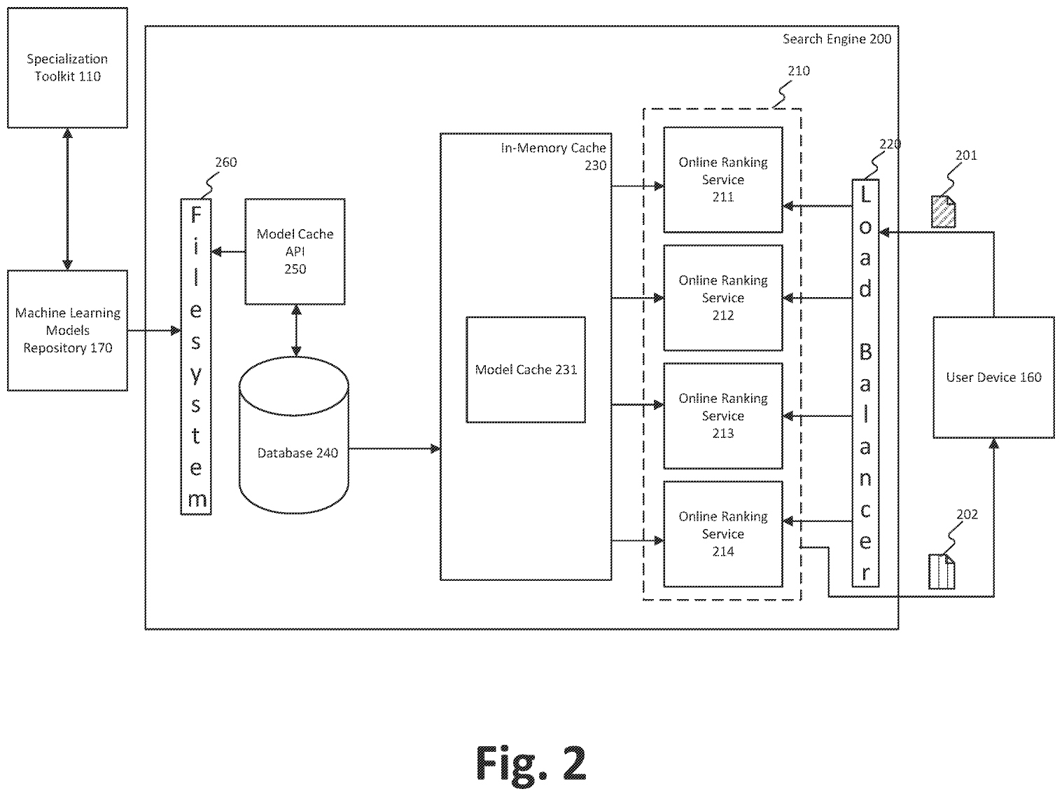

Specialization system 100 may determine expertise of service providers (e.g., service providers 131) accessible through a service provider search system (e.g., search engine 200 of FIG. 2). Specialization system 100 may function as the foundational layer of search engine 200 by providing service provider results with appropriate expertise to handle search requests to search engine 200. Specialization system 100 may evaluate various expertise of a service provider in the form of expertise, level of expertise, and specialties to determine the relevant service providers to surface relevant service provider matching the search request requirements sent to search engine 200.

Specialization system 100 may determine and store the expertise of service providers as expertise 121 by processing data associated with encounters (e.g., encounters 132) between service providers (e.g., service providers 131) and individuals (e.g., individuals 133). For example, a specialization system used in the healthcare service industry may process the claims data of past encounters between healthcare providers and their patients to determine expertise of the healthcare providers.

Specialization system 100 may access service providers 131 and associated individuals 133 and encounters 132 between them using specialization toolkit 110. Specialization toolkit 110 may include multiple modules to determine expertise of a service provider in the form of kinds of expertise, level of expertise, and specialties of a service provider. Modules in specialization toolkit 110 may work independently or in a certain order to determine expertise of various forms of service providers 131 in population database 130.

As illustrated in FIG. 1, specialization toolkit 110 may include expertise module 111, condition tiering module 112, and sub-specialty module 113 to determine various expertise and various forms of expertise of service providers 131. Specialization toolkit 110 may retrieve the relevant data from data warehouse 120 to determine expertise using expertise module 111. In some embodiments, specialization toolkit 110 may utilize ML platform 140 to train a Machine Learning (ML) model to predict expertise of service providers.

The determined expertise information of service providers may be used to identify relevant service providers for a search query posted by a user of search engine 200 (as shown in FIG. 2). The relevancy of a service provider may depend on the relationship between the search query and the expertise of a service provider. The relationship may be determined based on the additional information provided by a user of search engine 200 as part of search request 201. In some embodiments, the additional information may include settings outside of search request 201 settings. The additional information may include default values. For example, the location setting for service provider search may default to current location or service providers within a set distance from current location. Specialization system 100 may determine expertise of service providers in various forms based on the type of search queries and additional information supplied to search engine 200. The various forms of expertise as requested by a user using search engine 200 may be determined by expertise module 111, condition tiering module 112, sub-specialty module 113 beforehand or dynamically upon search engine 200 receiving a search request. A detailed description of an example search engine 200 used for handling search requests for service providers is provided in FIG. 2 description below.

Expertise module 111 may be used to identify expertise of a service provider in handling a particular condition. Expertise module 111 may thus answer the question of service for a particular condition in a binary manner as “Yes” or “No.” In some embodiments, specialization system 100 may review and revise expertise of service providers upon occurrence of certain events. Events may include periodic triggers to revise expertise of service providers at regular intervals. In some embodiments, introduction of new service provider(s) into population database 130 may trigger an event for specialization system 100 to determine their expertise. In some embodiments, search for service providers using search engine 200 may trigger events to determine expertise of service providers.

Specialization system 100 may offer configuration variables to evaluate expertise of service providers in terms of work performed on conditions. A user may set configuration variables using configuration file 150. Expertise module 111 may use Machine Learning (ML) models called condition models that may link service providers to conditions they can work on. Condition models may predict various conditions currently not handled by service provider to be part of service provider expertise. Expertise module 111 may interact with ML platform 140 to trigger condition models to determine the link between conditions (e.g., conditions 124) and service providers 131. ML platform 140 may trigger different condition models for each condition or set of conditions. ML models determining the links may store them in expertise 121.

Expertise module 111 may identify conditions handled by service providers by reviewing encounters (e.g., encounters 132) between service providers (e.g., service providers 131) and individuals (e.g., individuals 133) in need of services. A separate ML model may be trained for each of the many conditions handled by a service provider. Specialization system 100 may order the ML models for each condition in priority based on the number of encounters for service related to each condition. In some embodiments, ML models may be ordered based on the number of successful encounters. ML models may be run in the order they are sorted in determining expertise of service providers.

Expertise module 111 may prepare training data for condition models in three steps. In step 1, expertise module 111 may clean the data related to encounters (e.g., encounters 132) between service providers (service providers 131) and individuals (e.g., individuals 133) to identify top conditions treated by each service provider. For example, in a healthcare setting, claims data listing past encounters between healthcare providers and patients may be parsed to identify diagnosed conditions handled by service providers. Expertise module 111 may clean up data retrieved from various data resources before saving encounter information as encounters 132 in population database 130 and conditions 124 in data warehouse 120. Expertise module 111 may use data extractor 114, data transformer 115, and data loader 116 to retrieve and clean the data related to service providers to determine the expertise of service providers. Expertise module 111 may normalize data as part of data cleanup process.

Expertise module 111 may utilize ML models to identify conditions from service providers encounters with individuals. Expertise module 111 may provide as an input to ML models various procedures performed by service providers to predict the conditions handled by service providers. ML models may parse the text in the claims data and predict conditions that may be handled by service providers. ML models may predict conditions by determining service providers similar to a service provider with identified conditions.

In some embodiments, data cleanup process may involve determining non-relevant conditions associated with service providers. For example, in a healthcare setting, a claim for treating back pain by an orthopedist may also include diabetic treatment as it may be comorbidity, diabetes may be a non-relevant condition associated with the orthopedist. Such non-relevant conditions may be dropped by the expertise module 111 when cleaning up data for determining expertise. Expertise module 111 may request ML platform 140 to identify such non-relevant conditions by employing ML models. ML model may identify non-relevant conditions based on the procedures performed by service providers and conditions handled using performed procedures. In the above example, claims data inclusion of orthopedist recommendation of a physiotherapy procedure may make ML models determine that the recommended physiotherapy procedure is associated with back pain condition only. ML models may then predict diabetes as a non-relevant condition.

In step 2, expertise module 111 may label service providers in a binary manner for each of the identified conditions in step 1 as part of data cleanup process. The labels in a binary manner indicate whether a service provider may handle “x” condition or does not handle an “x” condition. Expertise module 111 may label all non-relevant conditions identified in step 1 as not handled by service providers associated with the non-relevant conditions. In some embodiments, expertise module 111 may label “x” condition as handled if a certain criterion is met, such as number of encounters or number of successful encounters.

In step 3, expertise module 111 may utilize ML models of ML models repository 170 to model output probability that a service provider can treat “x” condition. The output probability may depend on the number of individuals of individuals 133 who had a condition “x” and were handled by the service provider. In some embodiments, the length of the presence of condition “x” on claims data of individuals of individuals 133 may be considered in determining the probability of the service provider handling “x” condition. Percentage probability of handling conditions in addition to binary labels to handle conditions may be considered as expertise information associated with service providers.

Expertise module 111 may retrieve data from a variety of data sources (e.g., external reviews of service providers, claims data, and healthcare records of individuals) and process data so that it may be used with the remainder of specialization system 100. Expertise module 111 may further include a data extractor 114, data transformer 115, and data loader 116 modules. Data extractor 114, data transformer 115 may work together to generate the data in population database 130. Data transformer 115 may connect the disparate data extracted by data sources by data extractor 114 and store it in population database 130.

Data extractor 114 may retrieve data from data sources, including data related to service providers 131, encounters 132, and individuals 133. Each of these data sources may represent a different type of data source. For example, data source may be a database similar to population database 130. Data source may represent structured data, such as healthcare records and claims data of individuals. In some embodiments, data sources may be flat files, such as reviews of service providers. Further, data sources may contain overlapping or completely disparate data sets. In some embodiments, data source may contain information about individuals 133, while other data sources may contain other related data. For example, other data sources may be various insurance claims and medical treatment data of the individuals 133. Data extractor 114 may interact with the various data sources, retrieve the relevant data, and provide that data to the data transformer 115.

Data transformer 115 may receive data from data extractor 114 and process the data into standard formats. In some embodiments, data transformer 115 may normalize data such as dates. For example, a data source for healthcare records may store dates in day-month-year format, while data source for claims data may store dates in year-month-day format. In this example, data transformer 115 may modify the data provided through data extractor 114 into a consistent date format. Accordingly, data transformer 115 may effectively clean the data provided through data extractor 114 so that all of the data, although originating from a variety of sources, has a consistent format. For example, claims data may include middle names of Individuals 133 but healthcare records may not include the middle names. In the second example, data transformer 115 may include the missing middle name in healthcare records.

Moreover, data transformer 115 may extract additional data points from the data sent by data extractor 114. For example, data transformer may process a date in year-month-day format by extracting separate data fields for the year, the month, and the day. Data transformer 115 may also perform other linear and non-linear transformations and extractions on categorical and numerical data, such as normalization and demeaning. Data transformer 115 may provide the transformed and/or extracted data to data loader 116. In some embodiments, data transformer 115 may store the transformed data in population database 130 for later use by data loader 116 and other modules of expertise module 111.

Data loader 116 may receive the normalized data from data transformer 115. Data loader 116 may merge the data into varying formats depending on the specific requirements of specialization system 100 and store the data in an appropriate storage mechanism such as population database 130.

Expertise module 111 may determine expertise of service providers based on any presence of work done to handle conditions of conditions 124. In some embodiments, a certain level of experience may be needed in handling conditions for service providers to be considered to have expertise related to handled conditions. The experience in handling conditions may include amount of work done to work performed for handling conditions and amount of time involved in working to handle conditions. In some embodiments, experience in handling conditions may be include number and type of processes followed by service providers of service providers 131 when working to handle conditions. In some embodiments, amount of time and work done in handling a condition may define level of expertise of service providers. Condition tiering module 112 may help determine level of expertise of service providers by analyzing history of work performed in handling conditions.

A service provider's level of expertise may be determined by grading identified expertise of service providers 131. Specialization system 100 may achieve gradation of expertise based on work performed by service providers. Condition tiering module 112 may determine expertise levels of service providers.

In some embodiments, service providers of service providers 131 levels of expertise may be mapped to similar service providers. In some embodiments, similarity of individuals of individuals 133 associated with service providers of service providers 131 may be used in determining expertise levels of a service provider in question. Similarity between individuals of individuals 133 may be based on the similarity of geographical regions of service providers of service providers 131 and the users accessing services of service providers 131.

Condition tiering module 112 may help further determine expertise of service providers by identifying each expertise of expertise 121 associated with conditions 124 on a spectrum in the range of generalist to specialist. Condition tiering module 112 may answer questions of the form “Is the service provider truly an expert in handling x condition?” For example, in a healthcare setting, the expertise module 111 may provide answers to a question such as “Can the orthopedist treat back pain” in the form of “Yes” or “No.” On the other hand, condition tiering module 112 may provide answers to the question “Is the orthopedist a generalist who treats back pain, shoulders, knees, everything?” or “Is the orthopedist truly specializing in back pain?” Condition tiering module 112 may determine and store levels of expertise as expertise levels 122 in data warehouse 120. Condition tiering module 112 may also store labels of specific expertise as determined by condition tiering module 112 in data warehouse 120. In some embodiments, condition tiering module 112 may store specific expertise labels for service providers of service providers 131 with levels of expertise exceeding a threshold level. Service providers with lower expertise levels may have default labels, such as “generalist.” In the above example, the orthopedist may be associated with an expertise label for “back pain” or “generalist.”

Condition tiering module 112 may help identify expertise levels of service providers by determining expertise on a continuous range spectrum. In some embodiments, the expertise levels are discrete values. Service providers levels of expertise may be used in identifying relevant expert service providers for a user querying search engine 200 to handle a certain condition or provide a certain procedure for a certain condition. Service providers 131 histories saved in the form of encounters 132 may help in determining expertise levels (e.g., expertise levels 122) of service providers 131. The history of individuals 133 may be needed in determining expertise 121 and expertise levels 122 of service providers 131. Both expertise 121 and expertise levels 122 of service providers 131 may be needed for responding to queries (e.g., search request 201) to search engine 200. For example, in a healthcare setting, a patient's medical history in the form of encounters 132 may be reviewed to determine that they need an orthopedist who is an expert in “lower back pain” even if the user searches for condition “back pain” in search engine 200 (as shown in FIG. 2).

Condition tiering module 112 may only be involved in determining service providers expertise levels (e.g., expertise levels after expertise module 111 determines service providers (of service providers 131) expertise in handling certain conditions (of conditions 124). In some embodiments, condition tiering module 112 may directly determine whether a service provider is a non-zero level expert in handling a condition. In some embodiments, expertise module 111 may not consider service providers to be experts until their expertise levels reach threshold level as identified by condition tiering module 112. Expertise threshold levels may differ between conditions of conditions 124. Expertise threshold levels may be user customizable and provided via a configuration file (e.g., configuration file 150). In some embodiments, expertise threshold levels may be automatically determined by a ML model of ML models repository 170. ML model may evaluate the quality of service provided by service providers and outcomes of the provided service in handling conditions of conditions 124 to determine expertise threshold level required for handling a certain condition. Expertise threshold levels may vary with conditions and with other additional information associated with service providers, such as their geographical location. For example, in a healthcare setting, a healthcare provider may be considered an expert in a rural region with fewer service providers but considered a generalist in an urban region with more service providers having specific capabilities to handle specific conditions.

Condition tiering module 112 may be triggered to identify expertise levels for specified conditions. Specified conditions may be determined from history of encounters (e.g., encounters 132) of a user querying search engine 200 (as shown in FIG. 2) with service providers (e.g., service providers 131). The definition of specified conditions may be configurable and may vary with conditions. Specified conditions may be configured using configuration variables set in a text configuration file (e.g., configuration file 150).

Condition tiering module 112 may be employed to make alternate recommendations to users querying search engine 200 (as shown in FIG. 2) for handling certain conditions. The history of encounters of users with service providers (of service providers 131) may be used to determine alternate recommendations. Alternate recommendations may require condition tiering module 112 to be engaged to identify service providers of specific expertise and level of expertise to be considered. For example, in a healthcare setting, a search request 201 by a user of search engine 200 for “back pain” may result in suggestion of service providers specializing in “lower back pain” as alternate recommendations in addition to experts for handling “back pain” condition. The alternate recommendations may be based on user's history including claim data associated with lower back pain.

Condition tiering module 112 may be employed in circumstances where expertise level is an important factor when searching for expert service providers. For example, condition tiering module 112 may determine a need for a second opinion and find a true expert in a field to provide to a user as a recommendation.

Condition tiering module 112 may be used when determining deep specialization of service providers 131 is beneficial. For example, in a healthcare setting, a deep specialization determination may be done to handle conditions such as chronic headaches, certain cancer types. Condition tiering module 112 may determine deep specialization of service providers 131 based on expertise with highest level values.

Expertise module 111 may train a ML model of ML models repository 170 to respond to questions about expertise of service providers in a binary manner. Unlike expertise module 111 binary labeling model of “Yes” or “No,” condition tiering module 112 provides a continuous range of labels to service providers of service providers 131. Condition tiering module 112 may achieve a continuous range of labeling by providing a probability percentage that service providers are specialists. Condition tiering module 112 may use an unsupervised machine learning model (of ML models repository 170) to determine probabilities of expertise of service providers 131.

In some embodiments, condition tiering module 112 may attach conditions of conditions 124 handled by service providers 131 as labels to service providers based on the probabilities determined by the unsupervised machine learning model. Condition tiering module 112 may attach “generalist” label to service providers of service providers 131 with expertise probability percentage below a threshold value. The attached labels may be used for validation of truthfulness of expertise of service providers. Specialization system 100 may conduct validation during identification of service providers of service providers 131 to respond to a search request 201 (as shown in FIG. 2) sent to search engine 200 (as shown in FIG. 2). The specialization system 100 may not use the labels for training the unsupervised machine model.

Sub-specialty module 113 may help identify various types of potential procedures provided by service providers 131 for handling conditions (e.g., conditions 124). In some embodiments, sub-specialty module 113 may help identify an initial set of expertise areas for a service provider. Sub-specialty module 113 may retrieve the specialty of service providers 131 based on the details provided by service providers 131 in third-party databases. For example, in a healthcare setting, a healthcare provider may provide their specialties at an initial stage to the National Plan and Provider Enumeration System (NPPES) database that can be parsed by sub-specialty module 113 to retrieve service provider specialties.

In some embodiments, sub-specialty module 113 may review the education, fellowships, and residencies to determine the initial set of expertise. Sub-specialty module 113 may review the history of service providers 131 encounters 132 to determine further expertise gained by service providers 131. For example, a healthcare provider performing procedures for treating various diagnosed conditions may be reviewed from an external claims database to determine expertise of the healthcare provider. Sub-specialty module 113 may review the volume of the procedures, or the conditions handled using procedures to determine the specialties.

In some embodiments, sub-specialty module 113 may be used to find the specifics within an expertise area associated with a service provider. Expertise module 111 may determine an expertise area of service providers 131, and sub-specialty module 113 may identify the sub-areas of specialty within the determined expertise area. Sub-specialty module 113 may work with expertise module 111 to determine the hierarchy of expertise specialties.

Sub-specialty module 113 may determine hierarchy of expertise specialties in three steps. In step 1, sub-specialty module 113 may clean up the historical data of past encounters 132 between service providers 131 and individuals 133 to determine top conditions for each service provider. In some embodiments, expertise module 111 and its components data extractor 114, data transformer 115, and data loader 116 may be used to clean up data.

In step 2, for each top condition, sub-specialty module 113 may validate conditions treated by the service provider. Validation of a condition may include determining if a service provider has the ability to handle the condition. In some embodiments, sub-specialty module 113 may set up calls between validators and service providers to validate conditions. Sub-specialty module 113 may use a robot call service to automate communication with service providers.

Labels identifying condition specialties may be stored as specialties (e.g., specialties 123) of service providers 131. Sub-specialty module 113 may generate condition specialties based on the condition treated by service providers. For example, in a healthcare setting, a healthcare provider identified as an expert to treat muscular pain may have additional labels for neck pain, tail bone pain, etc., showcasing specific sub-specialties of treatment that are offered for muscular pain. Validators or automated tools may generate labels of specialties of service providers 131. In some embodiments, information from standardization bodies or common industry knowledge may be used to create labels. Labels from standardization bodies may be based on the training and education achieved by service providers 131. For example, in a healthcare setting, an OB/GYN who does not deliver babies is labeled “gynecologist,” and the one who delivers babies is labeled “obstetrician.” These labels may be obtained by reviewing the encounter data of OB/GYN healthcare providers. OB/GYN healthcare providers may have other labels based on their training as identified by ABMS board certification, including maternal & fetal medicine, reproductive endocrinology & infertility, urogynecology, gynecologic oncology. Validators or automated tools may be used to obtain information about other labels.

In step 3, sub-specialty module 113 may use validated conditions and other specialties identified as input labels to build ML models predicting whether a service provider handles a particular condition. ML models may be built by training existing ML models in ML models repository 170. ML models build in step 3 may include Kullback-Leibler divergence models. The built ML models are stored in ML models repository 170 and managed by ML platform 140. ML models may be used for making predictions of conditions to be associated with new service providers added to service providers 131. In some embodiments, ML models may aid in determining the stratification of service providers within a domain and, in turn, determine the hierarchy of conditions specialties forming expertise hierarchy.

Specialization system 100 may identify the specialties that are important before determining the stratification of condition specialties. Specialization system 100 may request sub-specialty module 113 to identify the important conditions and build stratified specialties using classification models. The classification models may be used to determine the strata in which a particular service provider of service providers 131 falls in. Sub-specialty module 113 may use different models for identifying different stratified condition specialties of expertise. Sub-specialty module 113 generation of expertise hierarchy is explained using two healthcare domains, OB/GYN and ophthalmology. The example domains are used to describe how labels are created, and ML models are utilized to stratify the labels identifying the condition specialties to generate expertise hierarchy.

In gynecology domain, sub-specialty module 113 may create “gynecologist” and “obstetrician” first stratum labels by reviewing past encounters stored in external claims database. In order to create sub-specialty labels, ABMS Board Certifications may be used. The certification forms second stratum of the OB/GYN labels. Second strata of labels may be obtained by using automated validators and retrieving from third-party data sources hosting data. ML models of ML models repository 170 built in step 3 above may be used to further predict labels in first and second stratum. Further, information about training and fellowships may be used where certification information for sub-specialties is missing. Sub-specialty module 113 may parse external databases to retrieve the alternate training information.

Stratified sub-specialties in OB/GYN domain may include rules as defined by ML models built in step 3 above to predict labels for expertise hierarchy. For example, any OB/GYN who had an OB/GYN Board Cert, but not a Board Cert for any of the sub-specialties, nor any sub-specialty fellowship training may be labeled as “Generalist”; and any OB/GYN with a Board Cert for one of the four sub-specialties may include labels under that sub-specialty. In addition, OB/GYN based on their work may be labeled as “Gynecologist” or “Obstetrician.”

In some embodiments, labels identifying sub-specialties may be identified by parsing third-party data. For example, ophthalmologists are neither given any specialty certifications by a standardization body nor are their education and training clearly demarked into specific specialties. In such a case, ophthalmologists may provide their own sub-specialties to a third-party database that may be parsed to add labels defining sub-specialties. The data extractor 114, data transformer 115, data loader 116 may be used to extract data from the database, including ophthalmologist self-identified specialties.

The specialty labels retrieved from third-party data sources may be used to build a random forest classifier model. The specialty labels retrieved from third-party data sources may be combined with data accessed using validators in step 2 above to improve ML models to predict specialties of service providers 131. The classifier models may be used to validate whether the self-identified specialties match the condition specialties identified from work history associated with a service provider. The classifier models may also predict other sub-specialties not identified by a service provider by using information from similar service providers as identified by the model. In some embodiments, a binary classifier model may be used for each sub-specialty labels retrieved from third-party data sources. Such models may be used for finding the appropriate specialist using service provider search service.

The sub-specialty labels associated with a service provider and the constructed and trained machine learning model may be used to connect the conditions treated by service providers to specialty labels. Sub-specialty module 113 may parse the work history data of a new service provider using conditional model to determine conditions and supply them to a trained sub-specialty model to determine the sub-specialty labels.

Specialization toolkit 110 may rely on data warehouse 120 to determine expertise of service providers 131 and store the determined expertise as expertise 121. Specialization toolkit 110 may use conditions 124 to determine the expertise 121 of service providers 131. Data warehouse 120 may store conditions identified from historical data and store them as conditions 124. Specialization toolkit 110 may rely on historical data from external data sources and previously processed data stored as encounters 132 in population database 130.

As illustrated in FIG. 1, data warehouse 120 may also be storage for previously evaluated various expertise stored as expertise 121. Expertise 121 may include expertise determined by expertise module 111, and expertise levels 122 determined by condition tiering module 112. In some embodiments, expertise 121 may also include the definitions of expertise as defined in configuration file 150 and used by expertise module 111 to evaluate expertise of service providers 131. Expertise levels 122 may include additional information about expertise of service providers 131. Expertise levels 122 may be generated by specialization toolkit 110 from expertise 121 to identify the true experts of conditions 124 associated with service providers of service providers 131.

Data warehouse 120 may also include codes 125 as identified by service providers in their encounters 132 with individuals 133. Codes 125 may represent understanding of service providers 131 of conditions 124 presented by individuals 133. Codes 125 may represent summary of conditions of conditions 124 identified during encounters 132 between service providers 131 and individuals 133.

Specialization system 100 may use data extractor 114, data transformer 115, data loader 116 to identify codes present third-party data sources, such as claims data. Codes 125 may map to multiple conditions of conditions 124. For example, in a healthcare setting, various conditions associated with pain in the facial area may be diagnosed as migraine and given a single code, such as a diagnostic code from diagnostic codes database. In another scenario, various conditions may be considered secondary conditions by service providers. Only the primary condition may be mapped to a code. For example, in a healthcare setting, a service provider treating for back pain may recommend chiropractic service for back pain and physiotherapy for legs which may have been pain developed due to back pain. Specialization system 100 may determine diagnostic code associated with back pain primary condition.

In some embodiments, multiple codes may be part of a condition, but only one code may be considered as the primary. For example, in a healthcare setting, an orthopedist treating a back may also include a diagnostic code for diabetes treatment as it may be comorbidity.

Data warehouse 120 may include procedures 126 offered by service providers 131 to handle conditions 124 presented by individuals 133 during encounters 132. Procedures 126 may include tests to confirm the diagnosis presented in the form of codes 125. Specialization system 100 may identify procedures 126 by parsing data related to encounters between service providers and individuals seeking service. In some embodiments, encounters of encounters 132 associated with codes 125 may include the steps to handle and resolve conditions.

In some embodiments, multiple procedures may be mapped to a single code. For example, in a healthcare setting, a code for a back disc slip may include procedures in the form of an MRI test scan to confirm the diagnosis and physiotherapy for pain relief. In some embodiments, specialization system 100 may determine the volume of each procedure provided by service providers to determine the most relevant procedures for each condition of conditions 124 and code of codes 125.

In various embodiments, data warehouse 120 and population database 130 may take several different forms. For example, population database 130 may be an SQL database or NoSQL database, such as those developed by MICROSOFT™, REDIS, ORACLE™ CASSANDRA, MYSQL, various other types of databases, data returned by calling a web service, data returned by calling a computational function, sensor data, IoT devices, or various other data sources. Data warehouse 120 may store data that is used or generated during the operation of applications, such as expertise module 111. For example, if expertise module 111 is configured to generate expertise specific to service providers such as service providers 131, then data warehouse 120 may store service providers evaluated expertise as expertise 121. Similarly, if condition tiering module 112 is configured to provide expertise levels, condition tiering module 112 may retrieve previously generated expertise and other related data stored in data warehouse 120. In some embodiments, data warehouse 120 and population database 130 may be fed data from an external source, or the external source (e.g., server, database, sensors, IoT devices, etc.) may be a replacement. In some embodiments, population database 130 may be data storage for a distributed data processing system (e.g., Hadoop Distributed File System, Google File System, ClusterFS, and/or OneFS). Depending on the specific embodiment of population database 130, data loader 116 may optimize the data for storing and processing in population database 130.

In some embodiments, specialization system 100 may utilize configuration file 150 provided using user device 160 to determine the expertise 121, expertise levels 122, and specialties 123 of service providers 131. User device 160 may be a processor or a complete computing device, such as laptops, desktop computers, mobile devices, smart home appliances, IoT devices, etc. Configuration file 150 may include definitions of expertise, expertise levels, and specialties as requested by a user of user device 160. Configuration file 150 and other information may be provided to specialization system 100 over network 180.

Configuration file 150 may provide a definition of expertise by listing the field names in population database 130 and other names to use as filter criteria in extracting values for field names from population database 130. Configuration file 150 may be presented as name-value pairs used to define various expertise requested by a user of user device 160. Configuration file 150 may include a description of service providers of service providers 131, individuals of individuals 133 receiving service. In some embodiments, configuration file 150 may also include types of service as criteria for filtering service providers 131 and encounters 132 of individuals 133 with service providers 131.

Specialization system 100 may include a defined structure for configuration file 150, such as YAML. Structured files such as YAML files may help in defining and evaluating expertise. Specialization system 100 may evaluate expertise of service providers 131 by querying databases (such as population database 130) storing events (such as encounters 132) associated with service providers 131. For example, expertise of a healthcare provider in handling conditions may include reviewing the encounters of the doctor with their patients. Specialization system 100 may parse the configuration file 150 in YAML format to generate the parsing functions to review and extract the relevant information from historical encounters between service providers 131 and individuals 133.

Specialization system 100, after parsing a configuration file 150 and determining expertise, expertise level, and specialties, may store requested them in data warehouse 120. Specialization system 100 may use the stored various expertise to determine the similarity between previously determined expertise of service providers 131 and service providers of service providers 131 expertise in handling conditions listed in configuration file 150.

Specialization system 100 may provide a graphical user interface to define various expertise and generate a configuration file (e.g., configuration file 150). In some embodiments, specialization system 100 may provide various conditions previously defined by a user in a dropdown UI. A user may generate a configuration file by selecting conditions of expertise using a GUI. In some embodiments, specialization system 100 may allow editing of selected conditions by updating filters, such as time period of a condition or other characteristics of individuals 133 to consider in determining expertise of service providers 131. Specialization system 100 may also include the ability to store the revised expertise with new identifiers in data warehouse 120. The use of structured languages such as YAML to format configuration files may help with easy generation of requests for expertise determination.

Network 180 may take various forms. For example, network 180 may include or utilize the Internet, a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, or other types of network communications. In some embodiments, network 180 may include an on-premises (e.g., LAN) network, while in other embodiments, network 180 may include a virtualized (e.g., AWS™, Azure™, IBM Cloud™ etc.) network. Further, network 180 may in some embodiments be a hybrid on-premises and virtualized network, including components of both types of network architecture.

Specialization system 100 may also help in identifying matching cohorts of individuals 133. The cohorts may differ in their association or lack of association with any service provider of service providers 131. Specialization system 100 may identify cohorts as part of determining expertise of service providers. Specialization system 100 may consider two cohorts of individuals 133 to be similar if the determined expertise match between cohorts.

Specialization system 100 may begin matching cohorts by finding cohorts of individuals 133 with matching characteristics. For example, specialization system 100 may find matching cohorts of patients by finding patients with matching pre-existing conditions, gender, age. In some embodiments, specialization system 100 may require more than one matching characteristic to select individuals for a matching cohort. The matching characteristics and the order and method of comparison may be configurable using parameters. In some embodiments, a user of user device 160 may provide configuration file 150 with parameters for finding matching cohorts.

A matching cohort may be used in determining expertise when the other matching service provider is missing a cohort of individuals for determining expertise. In some embodiments, matching cohorts may also be used in determining service provider recommendations. For example, service providers used by a cohort may be recommended to a matching cohort as part of search engine 200's search query (e.g., search request 201 of FIG. 2) results.

The expertise information of service providers 131 determined by specialization system 100 may be used to identify relevant service providers for a search query (e.g., search request 201 of FIG. 2) posted by a user of a service provider search system (e.g., search engine 200 of FIG. 2). The relevancy of a service provider may depend on the relationship between the search query and expertise of service provider of service providers 131. The relationship may be determined based on the additional information provided by a user of search engine 200. A detailed description of search engine 200 utilization of specialization system 100 to identify the relevant service providers with the appropriate expertise is presented in FIG. 2 description below.

FIG. 2 is a block diagram of an exemplary search engine 200, according to some embodiments of the present disclosure. As illustrated in FIG. 2, the internals of a search engine 200, which includes an online ranking service 210, may help in preparing a recommended list of service providers in response to search request 201. Preparation of list of service provider output 202 may include ordered listing and grouping of service providers.

Specialization system 100 may identify an appropriate specialist service provider based on a search request (e.g., search request 201) sent from a user device (e.g., user device 160) by a user. The search request 201 may vary based on the search terms and filters utilized in service provider search system (e.g., search engine 200). For example, a user of search engine 200 may search for a condition that needs to be handled, and the search engine 200 identifies specialist service providers of service providers 131 (as shown in FIG. 1) with expertise in handling the queried condition. In another scenario, a search for an expert may result in identifying a true expert among specialist service providers of service providers 131.

A user may supply as part of the user query the condition to be worked on and the procedure to use for working on the condition. Search engine 200 may then forward the condition to specialization system 100 to retrieve the service providers of service providers 131 associated with queried condition and procedure. In some embodiments, search engine 200 may need to send additional information such as location of the user, so the relevant service providers selected by specialization system 100 (as shown in FIG. 1) are close to the location of the user.

In some embodiments, a user may not directly provide the condition and may be determined by the search engine. Search engine 200 may determine the exact condition to be addressed and the expertise level requirement based on a series of questions. For example, a new user of search engine 200 may need to answer certain questions to identify the appropriate service provider. In some embodiments, specialization system 100 may provide a generalist on initial queries and provide specialists on later queries. For example, a patient searching for eye pain may be first directed to a primary care physician (PCP). In some embodiments, the generalists may themselves acquire certain specialties. For example, a PCP who studied internal medicine may be recommended for only adults. In some embodiments, a generalist may be chosen based on the specialist acquired due to services offered to the users of search engine 200.

In some embodiments, search engine 200 may select and present service providers based on a particular procedure to handle a condition. When a particular procedure is requested, a specialist service provider may be selected based on specialist labels determined by sub-specialty module 113. In some embodiments, specialist labels of service providers may be from their training and/or education. For example, a request for a knee surgeon may not list orthopedic surgeons or general surgeons but specialist surgeons who either had fellowships in knee surgery or have conducted several knee surgery procedures.

A user of search engine 200 may search for service providers based on their ability to work on a particular condition. In some embodiments, a user may search for a service provider who can perform a particular procedure. For example, in a healthcare setting, search engine 200 may request specialization system 100 to review various treatments performed by a healthcare provider on patients visiting the healthcare provider's office to identify healthcare providers with the ability to perform a particular treatment. The particular procedure performed by a service provider may be associated with handling a particular condition.

In some embodiments, a user searching for service providers with expertise in performing particular procedure may do so in combination with the condition to work on. For example, in a healthcare setting, a condition such as lower back pain may be searched along with physiotherapy treatment or chiropractic service, resulting in surfacing healthcare providers with expertise in working on back pain condition and also treating the condition by performing selected treatments (i.e., physiotherapy and chiropractic service). In some embodiments, the selected procedures may act as specialties (specialties 123 of FIG. 1) associated with service providers (e.g., service providers 131 of FIG. 1).

In some embodiments, a user may search for a service provider with a particular specialty. The user may search for particular specialty in combination with condition to be handled and particular procedure to handle condition. In some embodiments, conditions handled by a service provider may become their specialties. Specialties may also be attained by formal education and training. The service provider who is considered an expert for working on a particular condition or perform a particular service or has a particular specialty may be presented as various filters search engine 200. A detailed description of components of search engine 200 used for searching relevant service providers in different manners is described below.

As illustrated in FIG. 2, search engine 200 may comprise the online ranking service 210 to help determine the ranked order of the service providers to be part of a list of service provider output 202 shared with a user. The online ranking service 210 may be replicated multiple times across multiple computers of a cloud computing service (not shown in the figure). The multiple instances 211-214 of online ranking service 210 may help with handling multiple users' queries simultaneously. Specialization system 100 (not shown in the figure) may receive search request 201 and may delegate the online ranking service 210 to help determine the recommended list of service provider output 202.

The search engine 200 may also include a load balancer 220 to manage load of users' queries sent to the online ranking service 210. Load balancer 220 may manage the users' query load by algorithmically selecting an online ranking service instance of online ranking service instances 211-214. For example, load balancer 220 may receive search request 201 from user device 160 and forward it to online ranking service instance 211. In some embodiments, load balancer 220 may go through a round-robin process to forward the user queries to online ranking service instances 211-214. In some embodiments, online ranking service instances 211-214 may each handle different types of user queries. The type of query may be determined by load balancer 220.

The ranking method followed by online ranking service 210 may depend on the determined type of search request 201. In some embodiments, the ranked results generated by a set of online ranking service instances may be combined together by another set of online ranking service instances. For example, an online ranking service instance may rank based on the quality of healthcare provided, and another instance may rank based on the efficiency of the health care provider, and a third online ranking service may create composite ranks based on the ranking of service providers based on quality and efficiency.

Online ranking service 210 may utilize ML models to rank service providers. The online ranking service 210 may obtain the service providers through a set of ML models in ML models repository 170 and then rank them using another set of ML models in ML models repository 170. The ML models used for processing the identified service providers may reside in in-memory cache 230 for quick access. The ML models in in-memory cache 230 may be pre-selected or identified based on search request 201 sent by a user. Search engine 200 may include a model cache 231 to manage the ML models in the in-memory cache 230. In some embodiments, the model cache 231 may manage the models by maintaining a lookup table for different types of ML models. The model cache 231 may maintain and generate statistics about the ML models in in-memory cache 230. In some embodiments, the model cache 231 may only manage copies of models upon a user request. The model cache 231 may only include a single copy of each model in the in-memory cache 230. In some embodiments, the model cache 231 may also include multiple instances of the same ML models trained with different sets of data present in the database 240.

Specialization toolkit 110 may train ML models in ML models repository 170 before using them in search engine 200 to generate a recommended list of service provider output 202. Specialization toolkit 110 may train ML models based on expertise requested by a user using user device 160, as described in FIG. 1 description.

ML models in the in-memory cache 230 may be regularly copied from a key-value pair database 240 containing the trained ML models of ML models repository 170. Database 240 may access ML models in the ML models repository 170 using a model cache API 250. In some embodiments, the ML models repository 170 may be part of a file system 260. Database 240 may access ML models in ML models repository 170 to train the model at regular intervals. Database 240 supplies the trained ML models determined using ML models to in-memory cache 230 to be managed by model cache 331. The accessed ML models residing in database 240 and in-memory cache 230 may be utilized by both online ranking service 210 and other services that are part of specialization system 100.

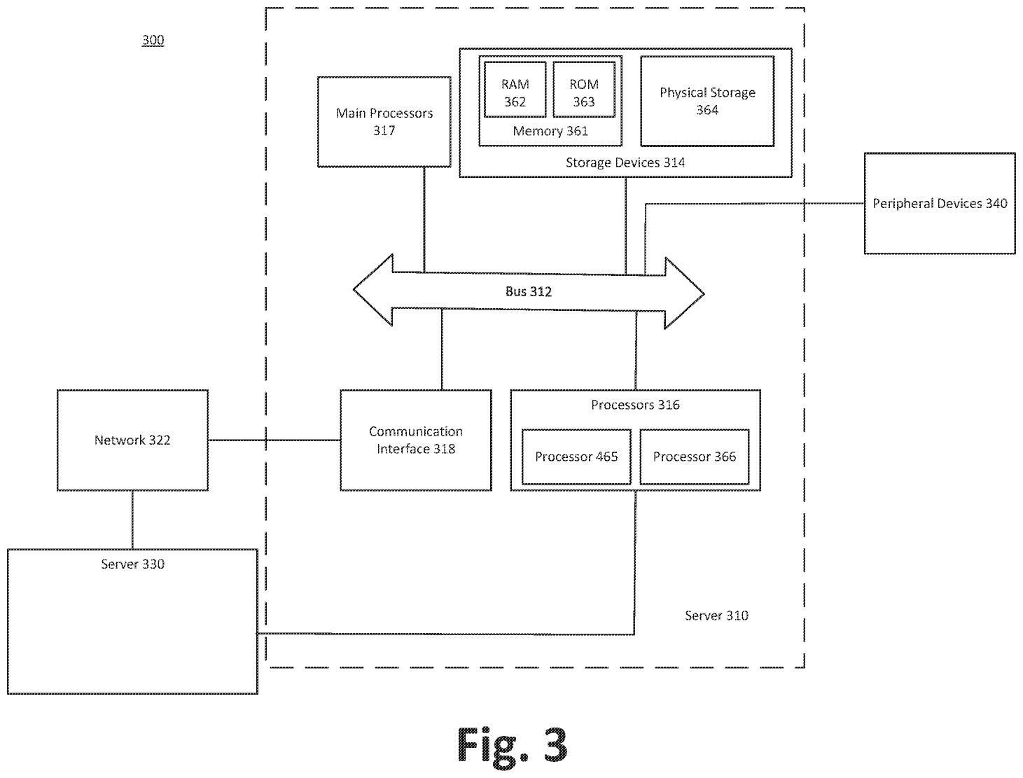

FIG. 3 illustrates a schematic diagram of an exemplary server of a distributed system, according to some embodiments of the present disclosure. According to FIG. 3, server 310 of distributed computing system 300 comprises a bus 312 or other communication mechanisms for communicating information, one or more processors 316 communicatively coupled with bus 312 for processing information, and one or more main processors 317 communicatively coupled with bus 312 for processing information. Processors 316 can be, for example, one or more microprocessors. In some embodiments, one or more processors 316 comprises processor 365 and processor 366, and processor 365 and processor 366 are connected via an inter-chip interconnect of an interconnect topology. Main processors 317 can be, for example, central processing units (“CPUs”).

Server 310 can transmit data to or communicate with another server 430 through a network 322. Network 322 can be a local network, an internet service provider, Internet, or any combination thereof. Communication interface 318 of server 310 is connected to network 322, which can enable communication with server 330. In addition, server 310 can be coupled via bus 312 to peripheral devices 340, which comprises displays (e.g., cathode ray tube (CRT), liquid crystal display (LCD), touch screen, etc.) and input devices (e.g., keyboard, mouse, soft keypad, etc.).

Server 310 can be implemented using customized hard-wired logic, one or more ASICs or FPGAs, firmware, or program logic that in combination with the server causes server 310 to be a special-purpose machine.

Server 310 further comprises storage devices 314, which may include memory 361 and physical storage 364 (e.g., hard drive, solid-state drive, etc.). Memory 361 may include random access memory (RAM) 362 and read-only memory (ROM) 363. Storage devices 314 can be communicatively coupled with processors 316 and main processors 317 via bus 312. Storage devices 314 may include a main memory, which can be used for storing temporary variables or other intermediate information during execution of instructions to be executed by processors 316 and main processors 317. Such instructions, after being stored in non-transitory storage media accessible to processors 316 and main processors 317, render server 310 into a special-purpose machine that is customized to perform operations specified in the instructions. The term “non-transitory media” as used herein refers to any non-transitory media storing data or instructions that cause a machine to operate in a specific fashion. Such non-transitory media can comprise non-volatile media or volatile media. Non-transitory media include, for example, optical or magnetic disks, dynamic memory, a floppy disk, a flexible disk, hard disk, solid state drive, magnetic tape, or any other magnetic data storage medium, a CD-ROM, any other optical data storage medium, any physical medium with patterns of holes, a RAM, a PROM, and an EPROM, a FLASH-EPROM, NVRAM, flash memory, register, cache, any other memory chip or cartridge, and networked versions of the same.

Various forms of media can be involved in carrying one or more sequences of one or more instructions to processors 316 or main processors 317 for execution. For example, the instructions can initially be carried out on a magnetic disk or solid-state drive of a remote computer. The remote computer can load the instructions into its dynamic memory and send the instructions over a telephone line using a modem. A modem local to server 310 can receive the data on the telephone line and use an infra-red transmitter to convert the data to an infra-red signal. An infra-red detector can receive the data carried in the infra-red signal, and appropriate circuitry can place the data on bus 312. Bus 312 carries the data to the main memory within storage devices 314, from which processors 316 or main processors 317 retrieves and executes the instructions.

Specialization system 100 (as shown in FIG. 1) or one or more of its components may reside on either server 310 or 330 and may be executed by processors 316 or 317. Search engine 200 (as shown in FIG. 2) or one or more of its components may also reside on either server 310 or 330. In some embodiments, the components of specialization system 100 and/or search engine 200 may be spread across multiple servers 310 and 330. For example, specialization toolkit 110 components 111-113 may be executed on multiple servers. Similarly, online ranking service instances 211-214 may be maintained by multiple servers 310 and 330.

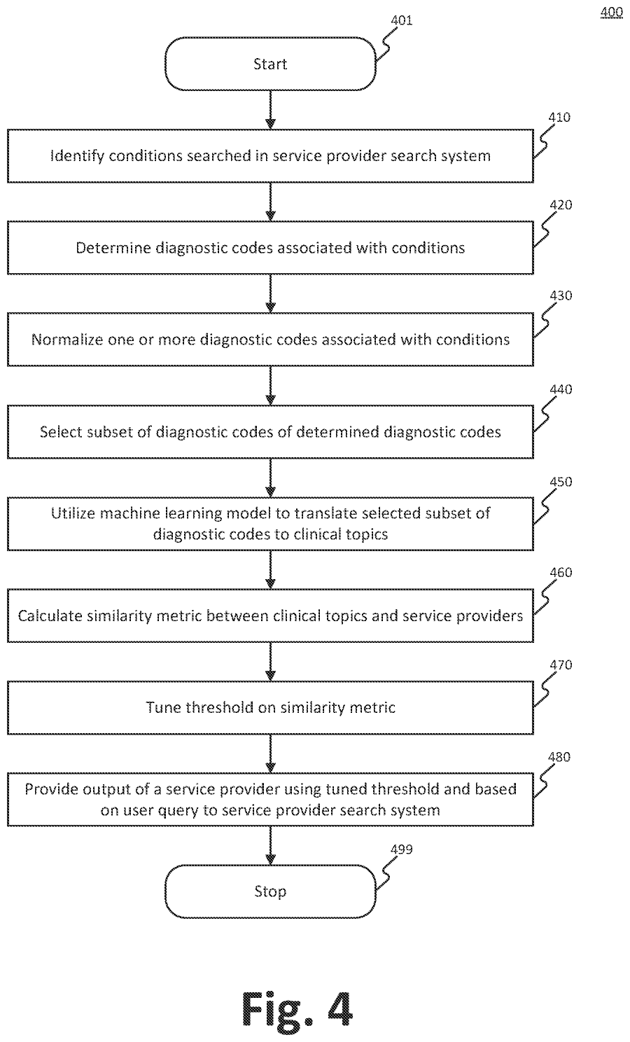

FIG. 4 is a flowchart showing an exemplary method for determining expertise of a service provider, according to some embodiments of the present disclosure. The steps of method 400 can be performed by, for example, specialization system 100 of FIG. 1 executing on or otherwise using the features of distributed computing system 300 of FIG. 3 for purposes of illustration. It is appreciated that the illustrated method 400 can be altered to modify the order of steps and to include additional steps.

In step 410, specialization system 100 may identify conditions searched using search engine 200 of FIG. 2. Search engine 200 may provide a filter field to include a condition as part of search request 201 (as shown in FIG. 2) sent to search engine 200. Specialization system 100 may parse the input for a condition and identify other related conditions stored in conditions 124. Specialization system 100 may also review encounters in encounters 132 to identify conditions associated with user of user device 160 that are handled by service providers. In some embodiments, when user of user device 160 is a new user, then encounters of a matching cohort of individuals of individuals 133 may be considered to identify conditions. The identified conditions are conditions associated with individuals of matching cohort that are already present in the specialization system 100. Specialization system 100 may review encounters 132 with any of service providers 131 to identify the conditions related to the condition included as part of search request 201. In some embodiments, specialization system 100 may request ML platform 140 to help identify other related conditions using a ML model of ML models repository 170 previously trained to determine conditions handled by service provides.

In step 420, specialization system 100 may determine codes of codes 125 (as shown in FIG. 1) and store them in data warehouse 120 as codes 125. In some embodiments, specialization system 100 may determine codes by reviewing encounters 132 associated with the user of user device 160.

In step 430, specialization system 100 may determine procedures (e.g., procedures 126) provided by service providers of service providers 131 to handle conditions based on service provider's diagnosis represented by codes (of codes 125 of FIG. 1). Procedures 126 may include tests to confirm the diagnosis presented in the form of codes determined in step 420. Procedures 126 may be determined by reviewing encounters of encounters 132 associated with codes determined in step 420. Procedures may be selected based on outcome analysis. Specialization system 100 may consider procedures associated with successful handling of conditions of conditions 124.

In step 440, specialization system 100 may normalize one or more codes of codes 125 associated with conditions of conditions 124 determined by specialization system 100. Specialization system 100 may normalize codes based on relationship between procedures associated with codes. In some embodiments, codes may be normalized based on their relation to the same conditions.

In step 450, specialization system 100 may select a subset of codes of determined codes (e.g., codes 125 of FIG. 1). Specialization system 100 may select codes for the service providers of service providers 131 with probability to handle conditions from step 410 more than the average probability of a set of similar service providers. In some embodiments, specialization system 100 may select a subset of service providers by identifying a subset of procedures that have most impactful outcome. Specialization system 100 may select codes associated with the identified subset of procedures.

Specialization system 100 may select a subset of the most common codes 125 identified in step 420. In some embodiments, a subset of codes 125 may be selected based on the location of the service providers of service providers 131 associated with the codes.

In step 460, specialization system 100 may utilize a ML model of ML models repository 170 to translate selected subset of codes 125 to topics of topics 127. Specialization system 100 may conduct the translation by reviewing external sources listing, such as Current Procedural Terminology (CPT) codes grouped under topics. CPT codes may represent procedures of procedures identified in step 430. Specialization system 100 may establish the mapping between codes and procedures of procedures 126 based on conditions 124 and codes 125.

In step 470, specialization system 100 may calculate similarity metric between topics and service providers using ML platform 140. ML platform 140 may determine the similarity metric by determining the codes of codes 125 associated with service providers 131 and identifying procedures provided under the codes with the codes of codes 125 under the topic.

ML platform 140 may determine similarity metric by determining expertise requirement of a user of search engine 200. ML platform 140 determines the requirement by reviewing the history of the user. ML platform 140, upon determination of expertise requirement, may identify a service provider that matches the expertise requirement.

In step 480, specialization system 100 may tune threshold on similarity metric to determine service providers of service providers 131 who may be accessible to user of user device 160 querying search engine 200.

Specialization system 100 may tune the similarity metric to improve the recall rate of the same service provider or a matching service provider as the top result in search output (e.g., service provider output 202 of FIG. 2). In some embodiments, specialization system 100 may also tune to improve the precision rate of the service providers chosen for condition included in search request 201. In some embodiments, specialization system 100 may further improve the precision rate by tuning to maintain the order of service provider results in search output.

In step 490, specialization system 100 may provide service provider output 202 based on search request 201. User of user device 160 may receive service provider output 202 as a list of service providers. Specialization system 100, upon completion of step 490, completes (step 499) executing method 400 on distributed computing system 300.

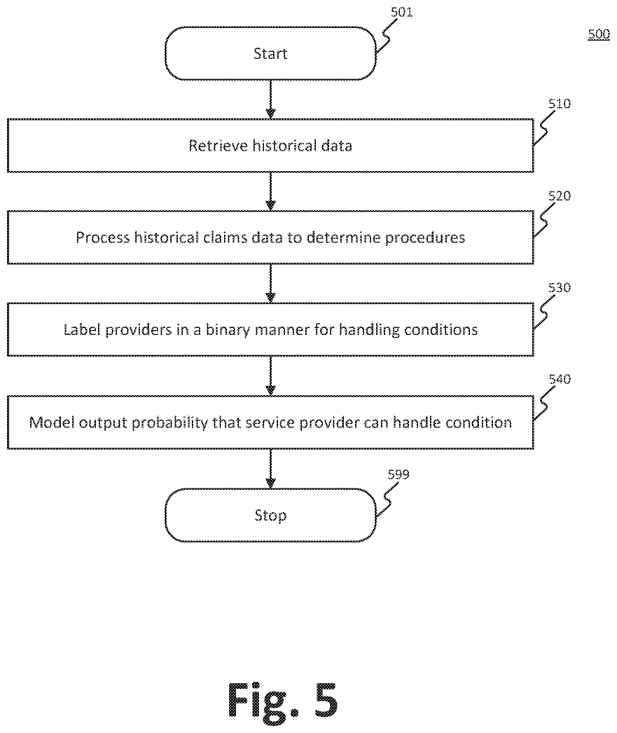

FIG. 5 is a flowchart showing an exemplary method for generating expertise of a service provider, according to some embodiments of the present disclosure. The steps of method 500 can be performed by, for example, expertise module 111 of FIG. 1 executing on or otherwise using the features of distributed computing system 300 of FIG. 3 for purposes of illustration. It is appreciated that the illustrated method 500 can be altered to modify the order of steps and to include additional steps.

In step 510, expertise module 111 may retrieve historical data, including encounters between service providers and individuals seeking their service. Expertise module may retrieve historical data from external sources over network 180. Expertise module 111 may retrieve historical data upon triggering of events. Expertise module 111 may consider a time interval or introduction of a new service provider as a triggering event. Specialization system 100 may allow customization of triggering events using a configuration file (e.g., configuration file 150). Configuration file 150 may include configurable variables to determine when to trigger events to parse historical data and what to parse and extract from historical data.

In step 520, expertise module 111 may process historical data to determine procedures recommended by a service provider. Expertise module 111 may parse the retrieved historical data from step 510 to access the procedures recommended by the service provider. Expertise module 111 may identify other related alternative procedures of procedures 126 that may be used to handle the same condition associated with a service provider. Expertise module 111 may request ML platform 140 to utilize a ML model of ML models repository 170 to determine related procedures. ML model may help identify related conditions and associated procedures by identifying service providers similar to the service provider in question. In some embodiments, ML model may identify a cohort of individuals matching the cohort served by the service provider in question and identify the service providers serving the matching cohort Service providers of matching cohort may be considered as similar service providers for determining related procedures.

In step 530, expertise module 111 may label service providers in a binary manner for handling conditions. In some embodiments, expertise module 111 may add binary labels upon evaluating success of a diagnosed condition and prescribed procedure to handle the condition. Expertise module 111 may determine the successful handling of a condition by reviewing historical data. Expertise module 111 may consider an encounter to be a success if a procedure to handle an associated condition does not repeat. In some embodiments, a procedure may be considered successful when an individual of individuals 133 (as shown in FIG. 1) does not appear post completion of procedure to handle a condition.

In step 540, expertise module 111 may model output probability that a service provider can handle a condition. Expertise module 111 may set the probability based on the number of times a condition is handled by the service provider in question. Expertise module 111 may determine the number based on the retrieved and processed historical data in steps 510 and 520. In some embodiments, expertise module 111 may only count situations where the condition was successfully handled by a service provider.

In some embodiments, expertise module 111 may set a probability of handling a condition by the service provider that has not been previously handled by the service provider. Expertise module 111 may set the probability based on procedures used by a service provider to handle conditions and the handled condition's relation to other conditions. In some embodiments, expertise module 111 may use a ML model of ML models repository 170 to predict the related conditions and accordingly the probability of the service provider handling the predicted related conditions. In some embodiments, expertise module 111 may use a ML model of ML models repository 170 to predict the probability of handling a new condition based on the closeness of the service provider in question and service providers of service providers 131 handling the new condition. In some embodiments, ML model may use the proximity of relationship between the individuals of individuals 133 associated with the new condition and the individuals associated with the service provider in question to predict probability in handling a condition. Expertise module 111, upon completion of step 540, completes (step 599) executing method 500 on distributed computing system 300.

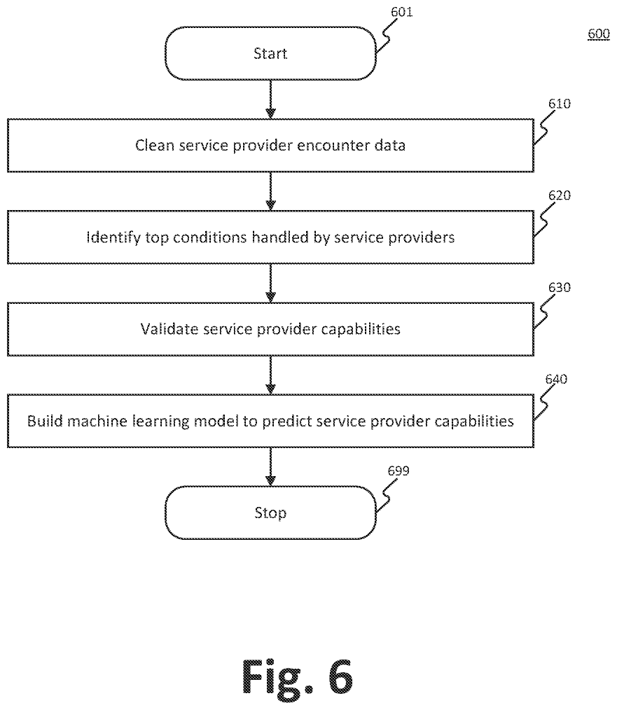

FIG. 6 is a flowchart showing an exemplary method for generating specialties of a service provider, according to some embodiments of the present disclosure. The steps of method 600 can be performed by, for example, specialization system 100 of FIG. 1 executing on or otherwise using the features of distributed computing system 300 of FIG. 3 for purposes of illustration. It is appreciated that the illustrated method 600 can be altered to modify the order of steps and to include additional steps.

In step 610, sub-specialty module 113 may clean up data related to encounters (e.g., encounters 132 of FIG. 1) of a service provider of service providers 131. Sub-specialty module 113 may receive the service provider in question from search engine 200 (as shown in FIG. 2). In some embodiments, specialization system 100 may determine a service provider identifier and send it to sub-specialty module 113 for determining additional expertise details in the form of sub-specialties of a service provider. The service provider identifier may be provided by expertise module 111 and condition tiering module 112 to determine other expertise of the service provider in question.

Sub-specialty module 113 may clean up the data by parsing historical data of encounters with service providers from an external data source over the network 180. Sub-specialty module 113 parses the historical data to identify the encounters of the service provider in question and then may store the encounter data as encounters 132 in population database 130. Sub-specialty module 113 may identify conditions diagnosed by the service provider during their encounters. Sub-specialty module 113 may store the identified conditions as conditions 124 in data warehouse 120.

In step 620, sub-specialty module 113 may identify top conditions handled by service provider in question from the conditions identified and saved as conditions 124 in data warehouse 120. Conditions of conditions 124 that appear the greatest number of times in the service provider encounters may be considered as top conditions. In some embodiments, sub-specialty module 113 may only consider conditions with the most appearances in a set period. The time period for identifying top conditions may be customizable. Specialization system 100 may allow the configuration of top condition determination time period in configuration file 150 (as shown in FIG. 1).

Sub-specialty module 113 may need to determine the primary conditions in each encounter associated with the service provider before identifying top conditions. Sub-specialty module 113 may identify top conditions from the primary conditions from each encounter. Sub-specialty module 113 may identify the topic (e.g., a clinical topic).

In step 630, sub-specialty module 113 may validate service provider capabilities by comparing the specialization information of service providers present on external data sources to those identified by sub-specialty module 113. Sub-specialty module 113 may use validators to confirm specialization obtained by validators and specialization determined from top conditions handled by a service provider. Validators may be automated bots generated and triggered by sub-specialty module 113 to determine the specializations posted by service providers on external data sources. For example, in a healthcare setting, healthcare providers may post the specializations they obtained from training and education on National Plan and Provider Enumeration System (NPPES) website. The bots triggered by sub-specialty module 113 may extract the specialization data posted on third-party websites. In some embodiments, bots may trigger a call between a validator and the service provider in question to find the specializations considered by the service provider.

Sub-specialty module 113 may determine topics (e.g., topics 127 of FIG. 1) encompassing various top conditions identified in step 620. In some embodiments, sub-specialty module 113 may determine topics 127 by identifying procedures of procedures 126 associated with top conditions identified in step 620. Sub-specialty module 113 may determine procedures by reviewing encounters of encounters 132 associated with top conditions identified in step 620. Sub-specialty module 113 may determine topics 127 by requesting external data resources with Current Procedural Terminology (CPT) codes to provide the encompassing topics for various procedures. In some embodiments, sub-specialty module 113 may need to map procedures listed in encounters associated with top conditions to procedures listed as part of codes database, such as CPT codes database. Sub-specialty module 113 may utilize ML models on ML platform 140 to determine the relevant CPT codes and encompassing topics based on procedures listed in encounters associated with top conditions of step 620. In some embodiments, ML model of ML models repository 170 may directly map the top conditions to topics.

In step 640, sub-specialty module 113 may build a ML model to predict a service provider's specialties in handling conditions of conditions 124. Sub-specialty module 113 may build a ML model by training a ML model of ML models repository 170 using ML platform 140. Sub-specialty module 113 may train ML model using validated specialization data obtained in step 630. Sub-specialty module 113 may use the trained ML model to predict specialization of other service providers. Sub-specialty module 113 may store the predicted specialties as specialties 123. Sub-specialty module 113, upon completion of step 640, completes (step 699) executing method 600 on distributed computing system 300.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Example embodiments are described above with reference to flowchart illustrations or block diagrams of methods, apparatus (systems) and computer program products. It will be understood that each block of the flowchart illustrations or block diagrams, and combinations of blocks in the flowchart illustrations or block diagrams, can be implemented by computer program product or instructions on a computer program product. These computer program instructions may be provided to a processor of a computer, or other programmable data processing apparatus to produce a machine, such that the instructions, which execute via the processor of the computer or other programmable data processing apparatus, create means for implementing the functions/acts specified in the flowchart or block diagram block or blocks.

These computer program instructions may also be stored in a computer readable medium that can direct one or more hardware processors of a computer, other programmable data processing apparatus, or other devices to function in a particular manner, such that the instructions stored in the computer readable medium form an article of manufacture including instructions that implement the function/act specified in the flowchart or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions that execute on the computer or other programmable apparatus provide processes for implementing the functions/acts specified in the flowchart or block diagram block or blocks.

Any combination of one or more computer readable medium(s) may be utilized. The computer readable medium may be a non-transitory computer readable storage medium. In the context of this document, a computer readable storage medium may be any tangible medium that can contain or store a program for use by or in connection with an instruction execution system, apparatus, or device.

Program code embodied on a computer readable medium may be transmitted using any appropriate medium, including but not limited to wireless, wireline, optical fiber cable, RF, IR, etc., or any suitable combination of the foregoing.

Computer program code for carrying out operations, for example, embodiments may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The flowchart and block diagrams in the figures illustrate examples of the architecture, functionality, and operation of possible implementations of systems, methods, and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts, or combinations of special purpose hardware and computer instructions.

It is understood that the described embodiments are not mutually exclusive, and elements, components, materials, or steps described in connection with one example embodiment may be combined with, or eliminated from, other embodiments in suitable ways to accomplish desired design objectives.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A non-transitory computer readable medium including instructions that are executable by one or more processors to cause a system to perform a method comprising:
identifying conditions searched in a service provider search system;
determining codes associated with the identified conditions, wherein each condition of the identified conditions is associated with one or more codes;
determining procedures provided by service providers available through the service provider search system, wherein each service provider of the services providers available through the service provider search system provides one or more procedures, wherein the procedures are associated with the determined codes;
normalizing the one or more codes associated with condition of the identified conditions;
selecting a subset of codes of the determined codes, wherein the selection is based on the popularity of procedures associated with the codes;
utilizing a machine learning model to translate the selected subset of codes to topics;
determining a similarity metric between the topics and the service providers, wherein service providers are those whose procedures are associated with code;
tuning threshold on the similarity metric; and
providing, using the tuned threshold, an output of a service provider based on a query by a user utilizing the service provider search system.

2. The non-transitory computer readable medium of claim 1, wherein identifying conditions further comprises:
processing the historical information of use of the plurality of service providers.

3. The non-transitory computer readable medium of claim 1, wherein selecting the subset of codes further comprises:
selecting the code for the service providers with more probability to treat than average probability of a set of similar service providers.

4. The non-transitory computer readable medium of claim 1, wherein selecting the subset of codes further comprises:
identifying a subset of procedures that have most impact on outcome; and
selecting the codes associated with the identified subset of treatment.

5. The non-transitory computer readable medium of claim 1, wherein determining procedures provided by service providers further comprises:
determining volume of each treatment of the procedures provided by each service provider of the one or more service providers.

6. The non-transitory computer readable medium of claim 1, wherein the machine learning model is a topical model.

7. The non-transitory computer readable medium of claim 1, wherein determining a similarity metric between the topics and the service providers available through the service provider search system further comprises:
determining expertise requirement of the user of the service provider search system, wherein the experiment requirement is based on service provider usage history of the user; and
determining a service provider with expertise level matching the expertise requirement.

8. The non-transitory computer readable medium of claim 1, wherein the instructions that are executable by one or more processors to cause the system to further perform:
determining the specialty of the service providers; and
selecting the service provider with specialties matching the query, wherein the procedures associated with a specialty match the procedures associated with a condition presented in the query.

9. The non-transitory computer readable medium of claim 1, wherein determining the specialty of service providers further comprises:
executing a machine learning model for each specialty, wherein a machine learning model is input the encounters of the service providers with the users of the service provider search system.

10. The non-transitory computer readable medium of claim 9, wherein the instructions that are executable by one or more processors to cause the system to further perform:
assigning default specialty labels for the service providers provided by the third-party database.

11. The non-transitory computer readable medium of claim 1, wherein tuning the threshold on the similarity metric further comprises:
improving recall rate of similar set of service providers for similar set of user queries.

12. The non-transitory computer readable medium of claim 1, wherein tuning the threshold on the similarity metric further comprises:
improving precision rate of same set of service providers for similar set of user queries.

13. The non-transitory computer readable medium of claim 1, wherein improving the precision rate of the same set of service providers includes maintaining the same order of the service providers.

14. The non-transitory computer readable medium of claim 1, wherein the instructions that are executable by one or more processors to cause the system to further perform:
receiving queries for specific services.

15. The non-transitory computer readable medium of claim 1 wherein the instructions that are executable by one or more processors to cause the system to further perform:
processing historic data from past;
determining procedures performed by a service provider to handle a condition;
generating a binary label for each condition based on the procedures; and
building a machine learning model; and
outputting probability of a service provider can handle a condition.

16. A method performed by a system for determining the expertise of service providers to match with users utilizing a service provider search system, the method comprising:
identifying conditions searched in a service provider search system;
determining codes associated with the identified conditions, wherein each condition of the identified conditions is associated with one or more codes;
determining procedures provided by service providers available through the service provider search system, wherein the procedures are associated with the determined codes;
normalizing the one or more codes associated with condition of the identified conditions;
selecting a subset of codes of the determined codes, wherein the selection is based on the popularity of procedures associated with the codes;
utilizing a machine learning model to translate the selected subset of codes to topics;
determining a similarity metric between the topics and the service providers, wherein service providers are those whose procedures are associated with code;
tuning the threshold on the similarity metric; and
providing, using the tuned threshold, an output of a service provider based on a query by a user utilizing the service provider search system.

17. The method of claim 16, wherein identifying conditions further comprises:
processing the historical information of use of the plurality of service providers.

18. The method of claim 16, wherein selecting the subset of codes further comprises:
selecting the code for the service providers with more probability to treat than average probability of a set of similar service providers.

19. The method of claim 16, determining procedures provided by service providers further comprises:
determining volume of each treatment of the procedures provided by each service provider of the one or more service providers.

20. A specialization system comprising:
one or more memory devices storing processor-executable instructions; and
one or more processors configured to execute instructions to cause the specialization system to perform:
identifying conditions searched in a service provider search system;
determining codes associated with the identified conditions, wherein each condition of the identified conditions is associated with one or more codes;
determining procedures provided by service providers available through the service provider search system, wherein the procedures are associated with the determined codes;
normalizing the one or more codes associated with condition of the identified conditions;
selecting a subset of codes of the determined codes, wherein the selection is based on the popularity of procedures associated with the codes;
utilizing a machine learning model to translate the selected subset of codes to topics;
determining a similarity metric between the topics and the service providers, wherein service providers are those whose procedures are associated with code;
tuning the threshold on the similarity metric; and
providing, using the tuned threshold, an output of a service provider based on a query by a user utilizing the service provider search system.
